(19) United States
(12) Patent Application Publication
(10) Pub. No.: US 2019/0275099 A1
(43) Pub. Date: Sep. 12, 2019

(54) Natural Treatment Regimen For Vaginal Yeast Infections

(71) Applicant: Nicole Tanner, Hyattsville, MD (US)

(72) Inventor: Nicole Tanner, Hyattsville, MD (US)

(21) Appl. No.: 15/912,575

(22) Filed: Mar. 6, 2018

(51) Int. Cl. A61K 36/889; A61K 36/61; A61K 36/58; A61K 36/73; A61K 9/00; A61K 9/02; A61P 31/10; A61P 15/02

(52) U.S. Cl. CPC A61K 36/889; A61K 36/61; A61K 36/58; A61P 15/02; A61K 9/0036; A61K 9/02; A61P 31/10; A61K 36/73

(57) ABSTRACT

A treatment regimen for an intravaginal yeast infection that comprises an application device to insert into a vagina comprised of a medication formulation. The medication formulation comprises a liquid compound comprised by percentage of 55%±5% coconut oil, 20%±2.5% tea tree or neem oil, and 25%±2.5% apple cider vinegar. The coconut oil is heated to between 100° F. and 115° F., with the tea tree or neem oil and the apple cider vinegar then mixed in to form the liquid compound.

### CROSS-REFERENCE TO RELATED APPLICATION(S)

Not applicable.

### BACKGROUND OF THE INVENTION

### 1. Field of Invention

The present invention relates to the field of natural medical treatments, more particularly to a natural treatment regimen for vaginal yeast infections.

### 2. Description of Related Art

Virtually all women will suffer from a yeast infection at some point in their lives. Vaginal yeast infection, also known as candidal vulvovaginitis and vaginal thrush, occur due to excessive growth of yeast in the vagina resulting in irritation. Symptom can include vaginal itching, burning with urination, vaginal discharge, pain with sex, and redness around the vagina. These symptoms can worsen just prior to periods.

Excessive growth of Candida or other yeasts causes vaginal yeast infections. Candia yeast is normally present in the vagina in a small amount, and contrary to some's belief, yeast infections are not classified as a sexually transmitted disease. However, it may occur more often in those who are frequently sexually active.

Noted risk factors include taking antibiotics, pregnancy, diabetes, and HIV/AIDS. Frequent sex has been associated with an increased risk, and a diet high in simple sugar. Tight clothing, type of underwear, and personal hygiene do not appear to be factors, although many women believe otherwise.

Diagnosis can be accomplished by testing a vaginal discharge sample. Since symptoms are similar to that of the sexually transmitted infections, chlamydia and gonorrhea, testing these STDs may be recommended.

Treatments that have been found effective and typically recommended include:

Intravaginal agents: butoconazole, clotrimazole, miconazole, nystatin, tioconazole, and terconazole.

Oral medication: By mouth: fluconazole as a single dose, and for severe cases another dose after 3 days may be prescribed.

Short-course topical formulations (i.e., single dose and regimens of 1-3 days) can also effectively treat uncomplicated candidal vulvovaginitis. Topically applied azole drugs are more effective than nystatin, and treatment with azoles results in relief of symptoms and negative cultures in 80-90%/o of patients who complete therapy.

Creams and suppositories in this regimen are oil-based and might weaken latex condoms and diaphragms. Treatment for vagina thrush using antifungal medication is ineffective in up to 20% of cases. Treatment for thrush is considered to have failed if the symptoms do not clear within 7-14 days.

There are a number of other intravaginal treatment options including creams or douches. For many women, the available intravaginal options can cause prove problematic. The harsh chemicals of over the counters treatment options, such as douches, can contain harsh chemicals such as boric acid, hydrogen peroxide, or iodine.

Based on the foregoing, there is a need for additional vaginal yeast infection options. Options made from natural components and avoiding harsh chemicals or costly anti-fungal can prove effective and less irritating to sensitive vaginal tissues.

### SUMMARY OF THE INVENTION

A treatment regimen for an intravaginal yeast infection that comprises an application device to insert into a vagina comprised of a medication formulation. The medication formulation comprised of a liquid compound comprised by percentage of −55%±5% coconut oil, 20%±2.5% tea tree or neem oil, and 25%±2.5% apple cider vinegar. The coconut oil is heated to between 100° F. and 115° F., with the tea tree or neem oil and the apple cider vinegar then mixed in to form the liquid compound.

The treatment regimen for an intravaginal yeast infection, wherein the application device comprises a suppository containing the liquid compound.

The suppository further comprises cocoa butter, polyethylene glycol, hydrogels, and/or glycerinated gelatin.

The application device comprises a plastic syringe loaded with 20 ml to 30 ml of the liquid compound to insert and inject the liquid compound in the vagina.

The application device comprises an elongated cylindrical medicated tampon in a plastic tubular package with an injection plunger for expelling the medicated pad into the vagina, wherein the medicated tampon is wetted or soaked with the liquid compound.

The application device comprises a sea sponge cut to fit within a vagina and wetted or soaked with the liquid compound and then inserted within the vagina

The liquid compound in a 1000 ml sample comprises about 525 ml of coconut oil, about 210 ml of tea tree or neem oil, and 265 ml of apple cider vinegar, ±5 ml for each ingredient.

A treatment compound for an intravaginal yeast infection, comprising a medication formulation comprised of a liquid compound comprising 55%±5% coconut oil, 20%±2.5% tea tree or neem oil, and 25%±2.5% apple cider vinegar. The coconut oil is heated to between 100° F. and 115° F., with the tea tree or neem oil and the apple cider vinegar then mixed in to form the liquid compound.

The foregoing, and other features and advantages of the invention, will be apparent from the following, more particular description of the preferred embodiments of the invention, the accompanying drawings, and the claims.

### DETAILED DESCRIPTION OF PREFERRED EMBODIMENTS

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

Headings provided herein are for convenience and are not to be taken as limiting the disclosure in any way.

The enumerated listing of items does not imply that any or all of the items are mutually exclusive, unless expressly specified otherwise.

The terms “a”, “an” and “the” mean “one or more”, unless expressly specified otherwise.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

The present invention will now be described in detail with reference to embodiments thereof as illustrated in the accompanying drawings.

### FORMULATION

The novel treatments options disclosed herein center on a natural formulation that includes coconut oil, tea tree oil or neem oil, and organic apple cider vinegar. For a 1000 ml mixture, the following amounts can be used:

525 ml coconut oil

210 ml tea tree oil OR neem oil

265 ml apple cider vinegar

These amounts can vary by ±5 ml for each of the ingredients.

By percentage, a mixture of about 55%±5% coconut oil, 20%±2.5% tea tree oil OR neem oil, and 25%±2.5% apple cider vinegar.

In preparation, the coconut oil is heated to between 100° F. and 1.15° F. and then the tea tree or neem oil and apple cider vinegar is mixed in and then the mixture is cooled.

After preparing the mixture, it can be cooled and applied intravaginal.

### Application

The mixture needs to be introduced intravaginally for treatment. Four applications methods have been contemplated and believed effective.

In a first embodiment, the formulation can be incorporated into a suppository. Suppositories are well known in the art for use to deliver a medicinal compound intravaginal by manual insertion. Such suppository can be made of a base compound formed into a dissolving capsule comprised of such as cocoa butter or a similar substitute, polyethylene glycol, hydrogels, and/or glycerinated gelatin. Cocoa butter may offer soothing effects to irritated vaginal tissue.

In a second embodiment, the formulation can be packaged in a medication loaded syringe holding a pre-measured amount of the treatment, such as a 20 ml to 30 ml pre-measured dose. A drawback of such an injection may be secretion of the medication requiring the use of a sanitary pad. The syringe can be individually packaged in a plastic or paper-based exterior package enclosing a plastic syringe with a closed end cap.

In a third embodiment, the formulation can be applied using an elongated cylindrical medicated tampon in a plastic tubular package with an injection plunger for expelling the medicated tampon into the vagina. In essence, the medicated tampon can be made essentially identically to a conventional tampon wetted or soaked with the medication mixture in a virtually identical plastic plunger to insert and then dispel the tampon into the vagina.

In a fourth embodiment, the formulation can be applied using a menstrual sponge (also known as a sea sponge tampon or period sponge) soaked with the medication and inserted manually by the user. In essence, the menstrual sponge can be made by trimming a sea sponge to the appropriate size and shape to fit within the vagina. The sponge can then be wetted by a pouring a quantity of the medication directly to the surface of the sponge or directly soaking the sponge in a container of the medication. The sponge itself can be washed and re-used, offering a totally natural treatment option devoid of any additional additives or disposable plastics, cloth, or paper products.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

I claim:

1. A treatment regimen for an intravaginal yeast infection, comprising:
an application device to insert into a vagina comprised of a medication formulation;
the medication formulation comprised of a liquid compound comprised by percentage of—
55%±5% coconut oil;
20%±2.5% tea tree or neem oil; and
25%±2.5% apple cider vinegar;
wherein the coconut oil is heated to between 100° F. and 115° F., with the tea tree or neem oil and the apple cider vinegar then mixed in to form the liquid compound.

2. The treatment regimen for an intravaginal yeast infection of claim 1, wherein the application device comprises a suppository containing the liquid compound.

3. The treatment regimen for an intravaginal yeast infection of claim 2, wherein the suppository further comprises cocoa butter, polyethylene glycol, hydrogels, and/or glycerinated gelatin.

4. The treatment regimen for an intravaginal yeast infection of claim 1, wherein the application device comprises a plastic syringe loaded with 20 ml to 30 ml of the liquid compound to insert and inject the liquid compound in the vagina.

5. The treatment regimen for an intravaginal yeast infection of claim 1, wherein the application device comprises an elongated cylindrical medicated tampon in a plastic tubular package with an injection plunger for expelling the medicated tampon into the vagina, wherein the medicated tampon is wetted or soaked with the liquid compound.

6. The treatment regimen for an intravaginal yeast infection of claim 1, wherein the application device comprises a sea sponge cut to fit within a vagina and wetted or soaked with the liquid compound and then inserted within the vagina.

7. The treatment regimen for an intravaginal yeast infection of claim 1, wherein the liquid compound in a 1000 ml sample comprises about 525 ml of coconut oil, about 210 ml of tea tree or neem oil, and 265 ml of apple cider vinegar, ±5 ml for each ingredient.

8. A treatment compound for an intravaginal yeast infection, comprising:
a medication formulation comprised of a liquid compound comprising—
55%±5% coconut oil;
20%±2.5% tea tree or neem oil; and
25%±2.5% apple cider vinegar;
wherein the coconut oil is heated to between 100° F. and 115° F., with the tea tree or neem oil and the apple cider vinegar then mixed in to form the liquid compound.
